(19) United States
(12) Patent Application Publication
(10) Pub. No.: US 2023/0248737 A1
(43) Pub. Date: Aug. 10, 2023

(54) COMPOSITIONS AND METHODS FOR TREATING PSYCHIATRIC DISORDERS OR SYMPTOMS THEREOF

(71) Applicant: PSYRX LTD., Holon (IL)

(72) Inventor: Asher HOLZER, Ra'anana (IL)

(21) Appl. No.: 18/003,561

(22) PCT Filed: Jun. 28, 2021
(86) PCT No.: PCT/IL2021/050787

§ 371 Date: Dec. 28, 2022

Related U.S. Application Data
(60) Provisional application No. 63/046,791, filed on Jul. 1, 2020.

(51) Int. Cl. A61K 31/55; A61K 31/137; A61K 31/138; A61P 25/24

(52) U.S. Cl. CPC A61K 31/55; A61K 31/137; A61K 31/138; A61P 25/24

(57) ABSTRACT

Abstract: The present invention provides pharmaceutical compositions and methods for treating psychiatric disorders, such as depression. The pharmaceutical compositions comprise a combination of ibogaine or derivative thereof and an antidepressant.

### FIELD OF THE INVENTION

The present invention relates to pharmaceutical compositions and methods for treating psychiatric disorders. In particular, the present invention relates to a combination of ibogaine and an anti-depressant drug for use in treating psychiatric disorders, such as depression.

### BACKGROUND

Psychiatric disorders include any mental disorder or illness that interferes with the way a person behaves, interacts with others, and/or functions in daily life. The exact cause of most psychiatric disorders is not known. Mental health experts believe that psychiatric disorders typically result from a combination of genetic or inherited dispositions and a triggering event.

Depression, also called major depressive disorder, is a neuropsychiatric disorder jeopardizing an increasing number of the population worldwide. Depression is a significant determinant of quality of life and survival, accounting for approximately 50% of psychiatric consultations.

In order to be diagnosed with clinical depression, a person’s depressed mood or anhedonia must be present for at least two weeks and be accompanied by other symptoms including feeling tired, sad, irritable, lazy, unmotivated, and apathetic. Other symptoms include an overwhelming fear or inability to feel emotion (emptiness), a decreased amount of pleasure in all or almost all daily activities, a change in weight, either loss or gain, disturbed sleep patterns including insomnia, loss of REM sleep, or excessive sleep. Clinically depressed patients can also have a psychomotor agitation or retardation, fatigue, difficulty concentrating, among others.

Various types and combinations of treatments may have to be tried, before reaching successful results. One mode of treatment is a medication. Alternative treatments used for depression include exercise and the use of vitamins, herbs, or other nutritional supplements. Antidepressants that relieve the symptoms of depression have been available for several decades.

Selective serotonin reuptake inhibitors (SSRIs) are a family of antidepressants considered to be the current standard of drug treatment. It is thought that one cause of depression is an inadequate amount of serotonin, a chemical used in the brain to transmit signals between neurons. SSRIs are said to work by preventing the reabsorption of serotonin by the nerve cell, thus maintaining the levels the brain needs to function effectively. This family of drugs includes fluoxetine (Prozac), paroxetine (Paxil, Seroxat), escitalopram (Lexapro), citalopram (Celexa), and sertraline (Zoloft).

Monoamine oxidase inhibitors (MAOIs), such as Nardil, are drugs that may be used if other antidepressant medications are ineffective. MAOIs are used to block the enzyme monoamine oxidase which breaks down neurotransmitters such as serotonin and norepinephrine (noradrenaline). Additional type of antidepressants are the tricyclic antidepressants and include, for example, amitriptyline and desipramine. Tricyclics block the reuptake of certain neurotransmitters such as norepinephrine (noradrenaline) and serotonin.

Other medications include norepinephrine reuptake inhibitors (NRIs), such as reboxetine (Edronax) that acts via norepinephrine, norepinephrine-dopamine reuptake inhibitors such as bupropion (Wellbutrin, Zyban) that inhibits the neuronal reuptake of dopamine and norepinephrine, and serotonin-norepinephrine reuptake inhibitors (SNRIs) such as venlafaxine (Effexor) and duloxetine (Cymbalta) that work on both noradrenaline and serotonin.

Many antidepressant drugs were developed for the treatment of depression. Yet, the depressed patients are often refractory to the antidepressant therapies. The limitations of the currently available antidepressants are related to their limited efficacy (only about 50% of the patients achieve remission), the delayed therapeutic effects and a great number of adverse/side effects, which includes headaches, constipation, weight changes, and sexual dysfunction. These issues highlight the need for better therapeutic agents that provide more efficacious and faster effects for the management of this disorder.

Ibogaine is a naturally occurring psychoactive substance found in plants in the family Apocynaceae such as Tabernanthe iboga, Voacanga africana, and Tabernaemontana undulata. Ibogaine-containing preparations are used for medicinal and ritual purposes within African spiritual traditions of certain tribes. Since the early 1960s, its apparent ability to reduce craving for psychoactive substances including alcohol, cocaine, methamphetamine, opiates, and nicotine has led to its use in detoxification treatments. The ibogaine is classified as a Schedule I drug by the Food and Drug Administration. In several other countries the ibogaine is legal and used to help recover from addictions. The use of ibogaine has been associated with serious side effects and death, however, there is a lack of clinical research.

There still remains an unmet need for safe and efficient pharmaceutical compositions and methods for treating psychiatric disorders such as depression.

### SUMMARY OF THE INVENTION

The present invention provides pharmaceutical compositions and methods for treating psychiatric diseases and disorders, in particular depression. The pharmaceutical compositions in some embodiments comprise ibogaine or derivatives thereof and at least one antidepressant.

It is now disclosed that the combination of ibogaine and antidepressants achieves improved efficacy. Without wishing to be bound by any theory or mechanism of action, it is postulated that the combination of ibogaine and antidepressants produces a synergistic effect in reducing symptoms of psychiatric disorders such as, depression. It is further postulated that the ibogaine and the antidepressant act in different pathways which together provides highly efficient therapy response. According to some embodiments, the combination of ibogaine and antidepressants acts synergistically, giving improved results over each of the active agents when used alone.

It is now disclosed that the combination of ibogaine and antidepressants reduces the time until onset of antidepressant response. Moreover, both ibogaine and antidepressant compound are used in reduced amounts which reduces side effects. The ibogaine is used in low doses which prevents its undesirable stimulant and hallucinogenic properties, as well as possible side effects such as nausea, vomiting, tremors, and increase of blood pressure.

According to one aspect, the present invention provides a pharmaceutical combination comprising: (1) ibogaine or a derivative thereof; and (2) at least one antidepressant.

According to some embodiments, the ibogaine is obtained from a natural source. According to additional embodiments, the ibogaine is a synthetic ibogaine.

According to some embodiments, the ibogaine derivative is selected from the group consisting of noribogaine, dihydroxyibogamine, dihydrocatharanthine, coronaridine, conopharyngine, conoflorine, catharanthine, iboxygaine, iboluteine, ibogamine, ibogaline, ibogaine, epiibogamine, isovoacangine, isovoacristine, 18-methoxycoronaridine (18-MC), kisantin, montanin, tabernanthine, tubotaiwine, voacristine, voacangine, voaluteine, and voacamine. Each possibility represents a separate embodiment of the invention.

According to certain embodiments, the ibogaine derivative is selected from the group consisting of coronaridine and voacangine.

According to some embodiments, the ibogaine derivative is 18-methoxycoronaridine (18-MC).

According to some embodiments, the ibogaine derivative is noribogaine.

According to some embodiments, the ibogaine is comprised of an extract or portion of a plant extract. According to some embodiments, the ibogaine is comprised of an extract or portion of a plant of the family Apocynaceae. According to certain embodiments, the ibogaine is comprised of an extract or portion of a Tabernanthe iboga, Voacanga africana, or Tabernaemontana undulata plant. According to specific embodiments, the ibogaine is comprised of an extract of an iboga plant extract.

According to some embodiments, the pharmaceutical combination comprises a plant extract comprising the ibogaine. According to certain embodiments, the pharmaceutical combination comprises both ibogaine and a derivative thereof.

According to certain embodiments, the pharmaceutical combination comprises two or more types of ibogaine or an ibogaine derivative.

According to some embodiments, the antidepressant is selected from the group consisting of monoamine oxidase (MAO) inhibitors, tricyclic antidepressants, serotonin reuptake inhibitors, selective norepinephrine reuptake inhibitors (SNRIs), aminoketones, serotonin antagonists, dopamine reuptake inhibitors, dual reuptake inhibitors, norepinephrine enhancers, serotonin activity enhancers, dopamine activity enhancers, and combinations thereof. Each possibility represents a separate embodiment of the invention.

According to some embodiments the antidepressant is selected from the group consisting of fluoxetine, bupropion, amitriptyline, clomipramine, desipramine, doxepin, imipramine hydrochloride, imipramine pamoate, maprotiline, nortriptyline, protriptyline, trimipramine, citalopram, escitalopram, moclobemide, fluvoxamine, paroxetine, sertraline, nefazodone, and trazodone. Each possibility represents a separate embodiment of the invention.

According to some embodiments, the antidepressant is Prozac.

According to certain embodiments, the antidepressant is selected from the group consisting of dopamine reuptake inhibitors, dopamine activity enhancers, norepinephrine enhancers, selective norepinephrine reuptake inhibitors (SNRIs), and combinations thereof.

According to some embodiments, the antidepressant is bupropion. According to certain exemplary embodiments, the antidepressant is selected from the group consisting of Aplenzin®, Budeprion SR®, Buproban®, Contrave®, Forfivo XL®, Wellbutrin SR®, Wellbutrin XL®, Zyban® and any combination thereof.

According to additional embodiments, the antidepressant is a selective serotonin reuptake inhibitor (SSRI). According to certain exemplary embodiments, the antidepressant is an SSRI selected from the group consisting of citalopram, escitalopram, fluoxetine, fluvoxamine, fluvoxamine CR, paroxetine, paroxetine CR, and sertraline.

According to some embodiments, the ibogaine or derivative thereof and the antidepressant are present in separate pharmaceutical compositions. According to certain embodiments, the ibogaine and/or derivative thereof, and the antidepressant are present in a single pharmaceutical composition.

According to an aspect, the present invention provides a pharmaceutical composition comprising the combination described herein and a carrier, diluent or excipient.

According to some embodiments, the pharmaceutical composition comprises ibogaine or derivative thereof in a dosage ranging from 5 mg to 1000 mg. According to some embodiments, the pharmaceutical composition comprises ibogaine or derivative thereof in a dosage ranging from 50 mg to 800 mg. According to some embodiments, the pharmaceutical composition comprises ibogaine or derivative thereof in a dosage ranging from 150 mg to 600 mg. According to some embodiments, the pharmaceutical composition comprises ibogaine or derivative thereof in a dosage ranging from 50 mg to 500 mg. According to some embodiments, the pharmaceutical composition comprises ibogaine or derivative thereof in a dosage ranging from 10 µg to 500 mg. According to some embodiments, the pharmaceutical composition comprises ibogaine or a derivative thereof in a dosage ranging from 50 µg to 200 mg. According to some embodiments, the pharmaceutical composition comprises ibogaine or a derivative thereof in a dosage ranging from 100 µg to 50 mg. According to some embodiments, the pharmaceutical composition comprises ibogaine or a derivative thereof in a dosage ranging from 300 µg to 20 mg.

According to some embodiments, the pharmaceutical composition comprises ibogaine or derivative thereof in a dosage of less than ⅕ of its recreational use. According to some embodiments, the pharmaceutical composition comprises ibogaine or derivative thereof in a dosage of less than ⅒ of its recreational use. According to some embodiments, the pharmaceutical composition comprises ibogaine or derivative thereof in a dosage of between ⅒ and 1/20 of its recreational use

According to some embodiments, the pharmaceutical composition comprises an antidepressant in a dosage ranging from 10 µg to 100 mg. According to some embodiments, the pharmaceutical composition comprises an antidepressant in a dosage ranging from 50 µg to 50 mg. According to some embodiments, the pharmaceutical composition comprises an antidepressant in a dosage ranging from 100 µg to 40 mg. According to some embodiments, the pharmaceutical composition comprises an antidepressant in a dosage ranging from 400 µg to 20 mg. According to some embodiments, the pharmaceutical composition comprises an antidepressant in a dosage ranging from 500 µg to 10 mg. According to some embodiments, the pharmaceutical composition comprises an antidepressant in a dosage ranging from 1 mg to 5 mg. According to additional embodiments, the pharmaceutical composition comprises an antidepressant in a dosage of less than 100 mg. According to additional embodiments, the pharmaceutical composition comprises an antidepressant in a dosage of less than 50 mg. According to additional embodiments, the pharmaceutical composition comprises an antidepressant in a dosage of less than 15 mg, 10 mg, 5 mg, 2 mg or 1 mg. Each possibility represents a separate embodiment of the invention.

According to specific embodiments, the pharmaceutical composition comprises ibogaine or derivative thereof in a dosage ranging from 500 µg and 1 g and an antidepressant as described herein in a dosage ranging from between 200 µg and 50 mg. According to specific embodiments, the pharmaceutical composition comprises ibogaine or derivative thereof in a dosage ranging from 100 µg and 500 mg and an antidepressant as described herein in a dosage ranging from between 100 µg and 20 mg. According to additional embodiments, the pharmaceutical composition comprises ibogaine or derivative thereof in a dosage ranging from 100 µg and 100 mg and an antidepressant as described herein in a dosage ranging from between 100 µg and 20 mg.

According to some embodiments, the pharmaceutical composition is formulated for oral administration. According to other embodiments, the pharmaceutical composition is formulated for injectable or inhalation administration. According to certain embodiments, the pharmaceutical composition is in a form of nasal drops or nasal spray.

According to some embodiments, the pharmaceutical composition is formulated in a dosage form selected from the group consisting of tablet, caplet, pill, capsule, pellets, granules, powder, lozenge, sachet, cachet, elixir, suspension, gel and dispersion. Each possibility represents a separate embodiment of the invention.

According to specific embodiments, the pharmaceutical composition is a tablet. According to certain embodiment, the tablet is suitable for once daily administration or twice a day administration to a subject.

According to various embodiments, the pharmaceutical composition further comprises at least one pharmaceutically acceptable excipient. According to particular embodiments, the pharmaceutically acceptable excipient comprises at least one of a binder, a filler, a surfactant, an anti-tacking agent, a glidant, a disintegrant, a diluent, a tonicity enhancing agent, a wetting agent, a buffering substance, a colorant, an antioxidant, a flavoring agent, a preservative, a stabilizer, or any combination thereof. Each possibility represents a separate embodiment.

According to some embodiments, the pharmaceutical composition is for use in the treatment of a disease, disorder or symptom amenable to treatment with an antidepressant.

According to some embodiments, the pharmaceutical composition is for use in treating a psychiatric disease or disorder.

According to some embodiments, the psychiatric disorder is selected from the group consisting of depression, schizophrenia, bipolar disorder, paranoia, panic disorder, anxiety, mania, post-traumatic stress disorder (PTSD), and obsessive-compulsive disorder.

According to some embodiments, the pharmaceutical composition is for use in treating PTSD. According to some embodiments, the pharmaceutical composition is for use in treating depression. According to certain exemplary embodiments, the pharmaceutical composition is for use in treating a treatment resistant depression (TRD).

According to some embodiments, the depression is selected from the group consisting of unipolar depression, major depressive disorder, psychotic depression, dysthymia, bipolar depression, atypical depression, melancholic depression, geriatric depression, treatment-resistant depression, antisocial personality disorder, single episodic, seasonal affective disorder, depression associated with menopause and recurrent major depressive disorder. Each possibility represents a separate embodiment of the invention.

According to some embodiments, pharmaceutical composition is for use in treating dissociative disorders, somatic symptom disorders, mood disorders, eating disorders, sleep disorders, disruptive disorders, neurocognitive disorders, schizophrenia, obsessive-compulsive disorders or personality disorders

According to some embodiments, the pharmaceutical composition is for use in treating obesity.

According to an additional aspect, the present invention provides a pharmaceutical combination of ibogaine or derivative thereof and at least one antidepressant.

The term “pharmaceutical combination” as used herein refers to either a pharmaceutical composition comprising one or more active pharmaceutical ingredient and one or more second therapeutic compounds or a pharmaceutical composition comprising an active pharmaceutical ingredient co-administered with a second therapeutic compound.

According to some embodiments, the ibogaine or derivative thereof and the at least one antidepressant are present in the same pharmaceutical composition. According to certain embodiments, the ibogaine or derivative thereof and the at least one antidepressant are present in separate pharmaceutical compositions.

In certain exemplary embodiments, the pharmaceutical combination comprises a pharmaceutical composition comprising an ibogaine or derivative thereof and a pharmaceutical composition comprising an antidepressant. The pharmaceutical compositions are as described herein.

According to an additional aspect, the present invention provides a method of treating depression comprising administering to a subject in need of such treatment a therapeutically effective amount of a pharmaceutical combination as described herein.

According to another aspect, the present invention provides a method of treating psychiatric disease or disorder comprising administering to a subject in need of such treatment a combination of therapeutically effective amounts of ibogaine or a derivative thereof and at least one antidepressant.

According to some embodiments, the neuropsychiatric disease or disorder is depression.

According to some embodiments, the subject is a mammal. According to certain embodiments, the subject is a human subject.

According to some embodiments, the subject was diagnosed as having depression by well recognized clinical set of criteria as outlined by DSM (Diagnostic and Statistical Manual of Mental Disorders), ICD (International Statistical Classification of Diseases and Related Health Problems or any other psychiatric classification system.

According to some embodiments, the treatment has no or minimal dissociative side effects upon administration to a subject.

According to some embodiments, the treatment reduces a depression symptom selected from the group consisting of neuropathic pain, sexual dysfunction, hopelessness, helplessness, anxiety, worries, memory problems, cognitive impairment, loss of feeling of pleasure (anhedonia), slowed movement, irritability, and lack of interest in personal care, such as poor adherence to medical or dietary regimens.

According to some embodiments, the treatment reduces a side effect selected from the group consisting of nausea, vomiting, dizziness, insomnia, sleepiness, trouble sleeping, abnormal dreams, constipation, sweating, dry mouth, yawning, tremor, gas, anxiety, eating disorders, agitation, abnormal vision, such as blurred vision or double vision, headache, and sexual dysfunction.

According to some embodiments, the treatment reduces the time until a clinical effect is shown.

According to some embodiments, the ibogaine or derivative thereof and the antidepressant are co-formulated. According to some embodiments, the ibogaine or derivative thereof and the antidepressant are present within a single pharmaceutical composition. According to some embodiments, the ibogaine or derivative thereof and the antidepressant are co-formulated in a pharmaceutical composition further comprising an excipient.

According to some embodiments, the method comprising administering a pharmaceutical composition comprising ibogaine or derivative thereof, and a pharmaceutical composition comprising an antidepressant.

According to some embodiments, the ibogaine or derivative thereof and the antidepressant are present in separate pharmaceutical compositions. According to certain exemplary embodiments, the ibogaine or derivative thereof and the antidepressant are formulated in separate orally available dosage form.

According to some embodiments, the method comprising administering a pharmaceutical composition comprising ibogaine or derivative thereof and an antidepressant.

According to some embodiments, the ibogaine or derivative thereof and the antidepressant are administered orally. According to alternative embodiments the administration is intravenous or intranasal. According to additional embodiments, the ibogaine or derivative thereof and the antidepressant are administered in different routes of administration.

According to some embodiments, the pharmaceutical composition is administered at least twice a day, once a day, twice a week or once a week. According to some embodiments, the pharmaceutical composition is administered at least once a day.

According to some embodiments, the method comprises administering the ibogaine or derivative thereof and the antidepressant at least once a day, at least twice a week, or at least once a week. According to certain embodiments, the method is carried out for at least a week, two weeks, a month or two months.

According to some embodiments, the administering of the ibogaine or a derivative thereof and the at least one antidepressant is carried out substantially simultaneously, concurrently, alternately, sequentially or successively. In some embodiments, the ibogaine or a derivative thereof and the at least one antidepressant are administered according to overlapping schedules.

According to particular embodiments, administering of the ibogaine is carried out prior to initial administration of the at least one antidepressant. According to some embodiments, the ibogaine or a derivative thereof and the at least one antidepressant are administered together. According to additional embodiments, the ibogaine or a derivative thereof and the at least one antidepressant are administered sequentially.

According to some embodiments, the method comprises one or two administering of high dosage of ibogaine or derivative thereof followed by continuous micro-dose treatment

According to some embodiments, the method comprises a step of administering a high dose of ibogaine on day 1 followed by administering reduced doses in the following days. According to some embodiments, the method comprises a step of daily administering of reduced doses of ibogaine for at least a week. According to some embodiments, the method comprises a step of administering a unit dosage form of 500-1000 mg of ibogaine on day 1 followed by maintenance dosages of less than 50 mg/day.

According to some embodiments, the method comprises administering the pharmaceutical composition at a daily dose comprising from about 0.1 to about 500 mg/day ibogaine and from about 0.5 to about 50 mg/day of an antidepressant. According to other embodiments, the method comprises administering said composition at a daily dose comprising from about 0.5 to 50 mg/day ibogaine and about 5 to about 10 mg/day of an antidepressant.

According to some embodiments, the antidepressant is administered in reduced amount compared to the standard dose. According to certain embodiments, the antidepressant is administered in a dose that is at least 10%, 20%, 30%, 40%, 50%, 60%, 70%, 80% or 90% lower than the presently approved dose. According to certain embodiments, the antidepressant is administered in a dose that is at least 10%, 20%, 30%, 40% or 50% lower than the standard dose. Each possibility represents a separate embodiment of the invention.

Any embodiment disclosed herein above can optionally be combined with the subject matter of one or any combination of another embodiment disclosed herein. For example, in some embodiments, the ibogaine or a derivative thereof is in a dosage ranging from 50 ug to 200 mg, and the antidepressant is in a dosage of less than 5 mg.

### DETAILED DESCRIPTION OF THE INVENTION

The present invention provides pharmaceutical compositions and methods for treating psychiatric diseases or disorder, in particular, depression. The methods of the invention comprise treating with a combination of ibogaine or derivative thereof and an antidepressant. The combination of the ibogaine and antidepressant in some embodiments provides a synergistic effect.

The combination therapy of ibogaine and an antidepressant is particularly advantageous in patients that suffer from the side effects of antidepressant that affect their efficiency, as the combination decreases side effects of antidepressants.

According to some embodiments, the combination of ibogaine and antidepressants acts synergistically, shortening the intake time (the duration it takes to start having clinical effects on patients). Usually, antidepressant drugs start having clinical effects only 45 days from first administration.

It is now disclosed that the combination of ibogaine and antidepressants acts synergistically, reducing the side effects usually associated with the standard of care today. For example, the combination reduces loss of appetite, loss of libido, sleeping disorders, and other adverse effects.

According to some embodiments, the combination of ibogaine and antidepressants acts synergistically, giving the ability to use a lower dosage of SSRI or other medication, yet having the same clinical efficacy.

As used herein, “synergy” or “synergistic” interchangeably refer to the combined effects of two active agents that are greater than their additive effects. Synergy can also be achieved by producing an efficacious effect with combined inefficacious doses of two active agents.

The terms “psychiatric disorder”, “mental disorder” and “mental illness” are used herein interchangeably and refer to mood disorders (e.g., depression of all forms and/or types, bipolar disorder, etc.), anxiety, anxiety disorders, psychotic disorders (e.g., schizophrenia, personality disorders), as well as other mental disorders.

Depression or depressive disorder is defined as psychoneurotic disorder characterized by mental and functional activity, sadness, reduction in activity, difficulty in thinking, loss of concentration, perturbations in appetite, sleeping, and feelings of dejection, hopelessness and generation of suicidal tendencies. It is a common and recurrent disorder causing significant morbidity and mortality worldwide.

Depression can vary in severity from mild to very severe. It is most often episodic but can be recurrent or chronic. Some people have only a single episode, with a full return to premorbid function. However, more than 50 percent of those who initially suffer a single major depressive episode eventually develop another.

Non limiting examples of depression include unipolar depression, major depressive disorder, bipolar disorder, psychotic depression, dysthymia, bipolar depression, atypical depression, melancholic depression, geriatric depression, treatment-resistant depression, single episodic, seasonal affective disorder, depression associated with menopause and recurrent major depressive disorder.

The term “bipolar disorder” as used herein refers to a mood disorder characterized by alternating periods of extreme moods. A person with bipolar disorder experiences cycling of moods that usually swing from being overly elated or irritable (mania) to sad and hopeless (depression) and then back again, with periods of normal mood in between.

The term “anxiety” as used herein refers to a condition characterized by feelings of worry, nervousness, unease, and/or tension, typically about an imminent event or something with an uncertain outcome. Symptoms of anxiety include, without limitation, fear, panic, heart palpitations, shortness of breath, fatigue, nausea, headaches, tachycardia, muscle weakness and/or tension, chest pain, stomach aches, sweating, trembling, pupillary dilation, panic attacks, and combinations thereof.

Dysthymia is characterized by depressed mood for at least 2 years as well as other symptoms like poor appetite or overeating, insomnia or hypersomnia, low energy or fatigue, low self-esteem, poor concentration or difficulty making decisions and feelings of hopelessness.

According to an aspect, the present invention provides a pharmaceutical composition comprising: (1) ibogaine or a derivative thereof; (2) an antidepressant, and a carrier, diluent or excipient.

The ibogaine compound is represented by formula I:

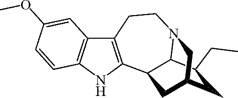 [SMILES: CC[C@H]1C[C@H]2C[C@H]3c4[nH]c5ccc(OC)cc5c4CC[N@@](C2)C13]

The present invention includes derivatives of ibogaine. The term “derivative” refers, for example, to compounds that are derived from another compound and maintain the same general structure as the compound from which they are derived. The term “derivative” when referred to ibogaine further includes modifications of the ibogaine compound. The present invention includes derivatives that retain the ibogaine activity.

According to some embodiments, the ibogaine is obtained from a natural source. According to certain embodiments, the ibogaine derivative is selected from the group consisting of coronaridine and voacangine.

According to some embodiments, the ibogaine derivative is 18-methoxycoronaridine (18-MC). According to additional embodiments, the ibogaine derivative is noribogaine.

Within the context of this disclosure “ibogaine derivative” can also mean a non-tryptamine molecule in a plant that produces ibogaine as a natural product, for example a molecule produced within the plant that influences the pharmacology of ibogaine or other tryptamines found in iboga.

According to additional embodiments, the ibogaine derivative is selected from the group consisting of dihydroxyibogamine, dihydrocatharanthine, coronaridine, conopharyngine, conoflorine, catharanthine, iboxygaine, iboluteine, ibogamine, ibogaline, epiibogamine, isovoacangine, isovoacristine, kisantin, montanin, noribogaine, tabernanthine, tubotaiwine, voacristine, voacangine, voaluteine, voacamine, 10-hydroxycoronaridine, 10-hydroxyheyneanine, 11-hydroxycoronaridine, 16-demethoxycarbonylvoacamine, 18-methoxycoronaridine, 19-hydroxyvenalstonine, 20-epivoacangarine, 3-(beta-hydroxyethyl)coronaridine, 3-hydroxyvoacangine, 3-oxo-11-methoxytabersonine, 3-oxocoronaridine, affinisine, ajmalicine, akuammicine, akuammidine, albiforanine, angustidine, angustine, angustoline, apparicine, condylocarpine, conoduramine, conodurine, conophyllidine, conophylline, coronaridine hydroxyindolenine, coronaridine pseudoindoxyl, cylindrocarpine, deplancheine, dregamine-1, dregamine-2, echitamidine, echitovenidine, ervadivaricatine B, ervatine, gabunamine, gabunine, geissoschizine, globospiramine, harmaline, harmine, heyneanine hydroxyindolenine, lbogamine-18-carboxylic acid, isositsirikine, jerantinine A, jerantinine B, jerantinine C, jerantinine D, jerantinine E, jerantinine F, koenimbine, kopsinine, lochnericine, lochneridine, minovin, N-methylibogaine, norfluorocurarine, pericyclivine, perivine, pyrifolidine, pyrifoline, refractine, rhazimine, sarcopharyngine, sewarine, stemmadenine, strictamin, tabernaelegantine A, tabernaelegantine B, tabernaemontanin, tabernamine, tabersonine, tetraphyllicine, tombozine, vallesiachotamine, vinburnine, vincadifformine, vindolinine, vinorine, voacangine hydroxyindolenine, voachalotine, voacorine, voafrine A, voafrine B, voaharine, voaphylline, voaphylline hydroxyindolenine, vobasine, vobtusine, yohimban, (+)20R-15,20-dihydro-cleavamine, (-)20S-15,20-dihydro-cleavamine, 14S,20R-velbanamine, 20R-1,2-dehydro-pseudo-aspidospermidine, 20S-hydroxy-1,2-dehydro-pseudoaspidospermidine, 19-epi-isovoacristine, 19-acetoxy-11-hydroxytabersonine, 19-hydroxy-ll-methoxytabersonine, 19-acetoxy-ll-methoxytabersonine, 19-epi-vindolinine, 19(S)-hydroxyibogamine, 19,20-epoxyconoduramine, 19-oxocoronaridine, 3R/S-ethoxy-19-epi-heyneanine, 3R/S-ethoxyheyneanine, desethyl-voacangine, gabonine, horhammericine, horhammerinine, iboquine, isovoacryptine, isovocangine, isovoacristine, kisantine, mehranine, pseudotabersonine, pyrifolidine, vinervine, desethyl-voacangine, hystrixnine, 19-oxovoacangine, and loganin.

According to some embodiments, the combination comprises ibogaine and ibogaine derivative as described herein. According to certain embodiments, the combination comprises 2 or more types of ibogaine or an ibogaine derivative. According to certain exemplary embodiments, the combination comprises ibogaine and noribogaine.

Exemplary molar ratios of the two types of ibogaine or ibogaine derivative include but are not limited to from 0.05:200 to 200:0.05, from 0.1:100 to 100:01, from 1:100 to 100:1, from 1:50 to 50:1, from 1:25 to 25:1, from 1:20 to 20:1, from 1:10 to 10:1, from 1:5 to 5:1, from 1:2 to 2:1, or 1:1.

According to some embodiments, the ibogaine or derivative thereof is in a dosage ranging from 1 µg to 1000 mg. According to some embodiments, the ibogaine or derivative thereof is in a dosage ranging from 50 µg to 750 mg. According to some embodiments, the ibogaine or derivative thereof is in a dosage ranging from 1 mg to 750 mg. According to some embodiments, the ibogaine or derivative thereof is in a dosage ranging from 100 mg to 700 mg. According to some embodiments, the ibogaine or derivative thereof is in a dosage ranging from 10 µg to 500 mg. According to some embodiments, the ibogaine or a derivative thereof is in a dosage ranging from 50 µg to 200 mg. According to some embodiments, the ibogaine or a derivative thereof is in a dosage ranging from 100 µg to 50 mg. According to some embodiments, the ibogaine or a derivative thereof is in a dosage ranging from 300 µg to 20 mg. According to some embodiments, the ibogaine or a derivative thereof is in a dosage ranging from 0.5 mg to 10 mg.

According to some embodiments, the ibogaine or derivative thereof is in a dosage of less than ⅕ of its recreational use. According to some embodiments, the ibogaine or derivative thereof is in a dosage of less than ⅒ of its recreational use. According to some embodiments, the ibogaine or derivative thereof is in a dosage of between ⅒ and 1/20 of its recreational use.

The term “antidepressant” refers to compounds as known in the art and are used to treat psychiatric disorders as described herein.

The antidepressant compounds used against depression are reported to be used also for treating additional conditions such as pain and anxiety syndromes. They have been grouped to several categories including Tricyclic antidepressants (TCAs), Selective serotonin-reuptake inhibitors (SSRIs), Monoamine oxidase inhibitors (MAOIs), Serotonin-norepinephrine reuptake inhibitor (SNRI) and Non-TCA antidepressants based on their mode of action.

Most of the antidepressants have been reported to possess adverse effects on the health of users. One of the drawbacks that current drugs have is that it usually takes several weeks until there is any clinical effect. The compositions described herein shorten the duration it takes to get significant clinical improvements.

According to some embodiments, the pharmaceutical compositions of the invention reduce the time until a clinical effect is shown.

Depression has different causes and forms including psychotic depression which characterized by severe depression, postpartum depression which characterized by perturbations in the levels of hormones and physical features after child birth and Seasonal Affective Disorder (SAD) which common mainly in the winter months with less sunlight.

One of the most important hypotheses for mood disorders relates to the alterations in the levels of biogenic amines. The occurrence of depression has been found to be associated with the alterations in the levels of biogenic amines in the brain such as NE, dopamine (DA) and epinephrine, indolamine, serotonin, 5-hydroxytryptamine (5-HT) and two catecholamines. It is now disclosed that adding certain level of ibogaine derivatives, in conjunction with existing antidepressant drugs has an influence on these molecules and their influence on the clinical condition of the patient. More specifically, the bioavailability and the effective level of serotonin has to do with the presence of some ibogaine derivatives.

The catecholamines are derived from a common precursor, the amino acid tyrosine. The first step in catecholamine synthesis is catalyzed by tyrosine hydroxylase in a reaction requiring oxygen as a co-substrate and tetrahydrobiopterin as a cofactor to synthesize dihydroxyphenylalanine (DOPA). Because tyrosine hydroxylase is rate-limiting for the synthesis of all three transmitters, its presence is a valuable criterion for identifying catecholaminergic neurons.

Dopamine is produced by the DOPA decarboxylase activity on DOPA. Although present in several brain regions, the major dopamine-containing area of the brain is the corpus striatum, which receives major input from the substantia nigra and plays an essential role in the coordination of body movements. Although dopamine does not readily cross the blood-brain barrier, its precursor, levodopa, does. Dopamine is also believed to be involved in motivation, reward, and reinforcement. Thus, it influences the patient state of mind and his depressive state. For example, cocaine and other addictive drugs act by stimulating the release of dopamine from specific brain areas. Once released, dopamine binds to specific dopamine receptors, as well as to some β-adrenergic receptors. It not only acts as a neurotransmitter in the central nervous system but also plays a role in some sympathetic ganglia. Dopamine is also used clinically to treat shock because it dilates renal arteries by activating dopamine receptors and increases cardiac output by activating β-adrenergic receptors in the heart.

Norepinephrine (also called noradrenaline) synthesis requires dopamine β-hydroxylase, which catalyzes the production of norepinephrine from dopamine. Dopamine is transported by vesicles into adrenergic terminals, where it is converted to norepinephrine. The most prominent class of neurons that synthesize norepinephrine is sympathetic ganglion cells, since norepinephrine is the major peripheral transmitter in this division of the visceral motor system. Norepinephrine is also the transmitter used by the locus coeruleus, where it influences sleep and wakefulness, attention, and feeding behavior.

Epinephrine (also called adrenaline) is present in the brain at lower levels than the other catecholamines. The enzyme that synthesizes epinephrine, phenylethanolamine-N-methyltransferase, is present only in epinephrine-secreting neurons. Epinephrine-containing neurons in the central nervous system are found in two groups in the rostral medulla.

All three catecholamines are removed by reuptake into nerve terminals or surrounding glial cells by Na+-dependent transporters. The two major enzymes involved in the catabolism of catecholamines are monoamine oxidase (MAO) and catechol O-methyltransferase (COMT). Both neurons and glia contain mitochondrial MAO and cytoplasmic COMT. Inhibitors of these enzymes, such as phenelzine and tranylcypromine, are used clinically as antidepressants.

Without wishing to be bound by a theory or mechanism, the presence of ibogaine in conjunction with some of the existing antidepressant drugs affects MAO and COMT activity.

Serotonin, also named 5-hydroxytryptamine (5-HT), is synthesized from the amino acid tryptophan, which is an essential dietary requirement. Tryptophan is taken up into neurons by a plasma membrane transporter and hydroxylated in a reaction catalyzed by the enzyme tryptophan-5-hydroxylase. As in the case of other biogenic amines, the synaptic effects of serotonin are terminated by transport back into serotonergic nerve terminals. The primary catabolic pathway is mediated by MAO. Serotonin is located in groups of neurons in the raphe region of the pons and upper brain stem, which have widespread projections to the forebrain and have been implicated in the regulation of sleep and wakefulness. A number of antipsychotic drugs used in the treatment of depression and anxiety are thought to act specifically on serotonergic neurons.

Without wishing to be bound by any theory or mechanism of action, ibogaine and ibogaine derivatives affect the serotonin level and function, although in different brain sites. The ibogaine and ibogaine derivatives influence the level, the potency and the affinity of serotonin transporters in a noncompetitive way to the other antidepressant drugs. Thus, ibogaine has a chaperoning effect that may increase the clinical outcomes such as more prolong effects, more immediate effects from starting intake, and may reduce the dose requirements of the existing antidepressant drugs, thus improve their efficacy and reduce the side effects and some of the adverse effects associated with the high dosages of those drugs.

According to some embodiments, the antidepressant is selected from the group consisting of: Clomipramine (Anafranil; serotonin-norepinephrine reuptake inhibitor), Trimipramine (Surmontil; serotonin-norepinephrine reuptake inhibitor), Amitriptyline (Saroten; serotonin-norepinephrine reuptake inhibitor), Nortriptyline (serotonin-norepinephrine reuptake inhibitor), Maprotiline (Ludiomil; serotonin-norepinephrine reuptake inhibitor), Fluoxetine (Fontex; serotonin reuptake inhibitor), Citalopram (Cipramil; serotonin reuptake inhibitor), Paroxetine (Seroxat; serotonin reuptake inhibitor), Sertraline (Zoloft; serotonin reuptake inhibitor), Fluvoxamine (Fevarin; serotonin reuptake inhibitor), Escitalopram (Cipralex; serotonin reuptake inhibitor), Moclobemide (Aurorix; MAO inhibitor).

The TCAs block the reuptake of both norepinephrine (NE) and serotonin (5HT). This phenomenon being the primary mechanism of action of antidepressants that changes in the physiological activity of neuro-receptors. The TCAs have also been reported to block muscarinic, alpha 1-adrenergic and histaminic receptors. These molecules may lead to occurrence of different side effects in patients. In some embodiments, the combination of ibogaine with these drugs reduces or even eliminate some of those side effects. In some embodiments, the combination of ibogaine with serotonin-reuptake inhibitors (SSRIs) blocks the reuptake of 5HT.

Non limiting examples of SSRIs include fluoxetine (Prozac, Selfemra), paroxetine (Paxil, Pexeva), sertraline (Zoloft), citalopram (Celexa) and escitalopram (Lexapro). SSRIs side effects include anxiety, sleep disturbances, sexual dysfunction (decreased libido, reduced pleasurably and reduction in arousal), and gastrointestinal disturbances.

A reciprocal relationship exists between serotonin and dopamine. While serotonin tends to inhibit sexual functioning, dopamine enhances sexual functioning (Shrma B, J Appl Biotechnol Bioeng. 2017;3(5):437-448). Without wishing to be bound by any theory or mechanism, the serotonin pathway descending from brain stem down the spinal cord to spinal neurons mediates various spinal reflexes that responsible for the sexual dysfunction in the form of ejaculation and orgasm problems. It has been reported that the enhanced serotonergic flow through this pathway inhibits sexual functioning. The serotonin’s negative effects on sexual functioning are mediated via 5-HT2 receptors.

MAOIs are generally prescribed in cases of atypical or drug resistant depression. These compounds contain a certain level of toxicity. Additional drug is moclobemide (manerix) which has been reported to be the first reversible inhibitor of monoamine oxidase A (RIMA). This molecule is found to be effective and safe. In some embodiments, the combination of those drugs with ibogaine increases their reversible inhibitory activity of monoamine oxidase A (RIMA).

Nefazodone (Serzone) acts like SSRIs which blocks the reuptake of 5HT and also has synaptic 5HT transmission activity. The SSRIs have very little or insignificant effect on the reuptake of other neurotransmitters. It has been shown that SSRIs does not display any activity on the muscarinic and histaminergic receptors which may results in minute anticholinergic (ACH) and sedative effects. The mechanisms of actions of antidepressants such as monoamine oxidase inhibitors (MAOIs), phenelzine (Nardil) and tranylcypromine (Parnate) associate with the inhibition of the enzymatic conversion of 5HT and NE into their corresponding antagonist of 5HT2 receptor, thereby reducing the stimulating effects similar to SSRIs. In some embodiments, the combination of (i) ibogaine and (ii) monoamine oxidase inhibitors (MAOIs), phenelzine or tranylcypromine, affects the inhibition of the enzymatic conversion of 5HT and NE into their corresponding antagonist of 5HT2 receptor, thereby enhances the reduction of the stimulating effects similar to SSRIs.

Nefazodone and trazodone (Desyrel) are antidepressants that act as antagonists of the α1 adrenergic receptors. According to some embodiments, the ibogaine increase the antagonistic activity of nefazodone and trazodone (Desyrel).

According to some embodiments, the combination of ibogaine with an antidepressant reduces side effects.

Side effects include but not limited to numbness and tingling in the arms and legs, headache, blurred vision, constipation or diarrhea, nausea, skin rash, swelling of the face and tongue, unexpected weight gain or loss, narrow dizziness or light headedness, confusion, difficulty in urinating, and dry mouth.

The antidepressants according to the invention further include but not limited to selective serotonin reuptake inhibitors [Citalopram (Celexa), Escitalopram (Lexapro, Cipralex), Paroxetine (Paxil, Seroxat), Fluoxetine (Prozac), Fluvoxamine (Luvox), Sertraline (Zoloft, Lustral)], serotonin-norepinephrine reuptake inhibitors [Duloxetine (Cymbalta), Milnacipran (Ixel, Savella), Venlafaxine (Effexor), Desvenlafaxine (Pristiq), Tramadol (Tramal, Ultram), Sibutramine (Meridia, Reductil)], serotonin antagonist and reuptake inhibitors [Etoperidone (Axiomin, Etonin), Lubazodone (YM-992, YM-35,995), Nefazodone (Serzone, Nefadar), Trazodone (Desyrel)], norepinephrine reuptake inhibitors [Reboxetine (Edronax), Viloxazine (Vivalan), Atomoxetine (Strattera)], norepinephrine-dopamine reuptake inhibitors [Bupropion (Wellbutrin, Zyban), Dexmethylphenidate (Focalin), Methylphenidate (Ritalin, Concerta)], norepinephrine-dopamine releasing agents [Amphetamine (Adderall), Dextroamphetamine (Dexedrine), Dextromethamphetamine (Desoxyn), Lisdexamfetamine (Vyvanse)], tricyclic antidepressants [Amitriptyline (Elavil, Endep), Clomipramine (Anafranil), Desipramine (Norpramin, Pertofrane), Dosulepin [Dothiepin] (Prothiaden), Doxepin (Adapin, Sinequan), Imipramine (Tofranil), Lofepramine (Feprapax, Gamanil, Lomont), Nortriptyline (Pamelor), Protriptyline (Vivactil), Trimipramine (Surmontil)], tetracyclic antidepressants [Amoxapine (Asendin), Maprotiline (Ludiomil), Mianserin (Bolvidon, Norval, Tolvon), Mirtazapine (Remeron)], monoamine oxidase inhibitors [Isocarboxazid (Marplan), Moclobemide (Aurorix, Manerix), Phenelzine (Nardil), Selegiline [L-Deprenyl] (Eldepryl, Zelapar, Emsam), Tranylcypromine (Parnate), Pirlindole (Pirazidol)], 5-HT_{1A} Receptor Agonists [Buspirone (Buspar), Tandospirone (Sediel), Vilazodone (Viibryd)], 5-HT2 Receptor Antagonists [Agomelatine (Valdoxan)], Nefazodone (Nefadar, Serzone), and selective Serotonin Reuptake Enhancers [Tianeptine].

According to some embodiments, the antidepressant is selected from the group consisting of amitriptyline, clomipramine, desipramine, doxepin, imipramine hydrochloride, imipramine Pamoate, maprotiline, nortriptyline, protriptyline, trimipramine, citalopram, fluoxetine, fluvoxamine, paroxetine, sertraline, nefazodone, and trazodone. Each possibility represents a separate embodiment of the invention.

According to certain embodiments, the antidepressant is Prozac.

According to some embodiments, the pharmaceutical combination comprises ibogaine or derivative thereof, and a monoamine oxidase (MAO) inhibitor. According to some embodiments, the pharmaceutical combination comprises ibogaine or derivative thereof, and a tricyclic antidepressant. According to some embodiments, the pharmaceutical combination comprises ibogaine or derivative thereof, and a selective norepinephrine reuptake inhibitor. According to some embodiments, the pharmaceutical combination comprises ibogaine or derivative thereof, and a dopamine reuptake inhibitor. According to some embodiments, the pharmaceutical combination comprises ibogaine or derivative thereof, and a norepinephrine enhancer. According to some embodiments, the pharmaceutical combination comprises ibogaine or derivative thereof, and a dopamine activity enhancer.

The active ingredient of Prozac is fluoxetine hydrochloride and is usually being administered orally as a tablet or a liquid. The standard dose is 20-60 mg orally per day. Prozac is usually being prescribed to depression, bulimia, obsessive compulsive disorder, panic disorder, and premenstrual dysphoric disorder.

According to additional embodiments, the antidepressant is bupropion.

According to an aspect the present invention provides a pharmaceutical combination comprising (1) ibogaine or derivative thereof; (2) bupropion, and a carrier, excipient, and diluent.

According to an aspect the present invention provides a pharmaceutical composition comprising (1) ibogaine or derivative thereof; (2) bupropion, and a carrier, excipient, and diluent.

According to some embodiments, the antidepressant is selected from the group consisting of citalopram (Celexa), escitalopram (Lexapro), fluoxetine (Prozac, Sarafem, Selfemra), fluvoxamine (Luvox), paroxetine (Paxil, Paxil CR, Pexeva), sertraline (Zoloft), vortioxetine (Trintellix, formerly known as Brintellix), and vilazodone (Viibryd).

A suitable dose of the antidepressant compound may be in the range recommended by the manufacturer or reported in the literature. According to some embodiments, the antidepressant agent is used at the low end of the range recommended by the manufacturer, or even below the range, in view of the synergistic benefits that can be achieved according to the present invention.

Non limiting examples of doses provided for certain antidepressants include: Amitryptiline: about 100-300 mg/day maintenance dose; Buproprion: from about 100 to about 300 mg/day; Citalopram: from about 5 to about 50 mg once/day; Clomipramine: typically about 100-250 mg/day maintenance dose; Duloxetine: from about 1 to about 30 mg once/day; Fluoxetine: from about 1 to about 80 mg, once/day; Fluvoxamine: from about 20 to about 500 mg once/day; Imipramine: about 100-300 mg/day maintenance dose; Isocarboxazid: about 10-20 mg/day maintenance dose; Maprotiline: about 100-200 mg/day maintenance dose; Mianserin: about 30-90 mg/day maintenance dose; Milnacipran: about 10 to about 100 mg once-twice/day; Mirtazapine: about 14-45 mg/day maintenance dose; Moclobemide: about 300-600 mg/day maintenance dose; Nefazodone: about 150-300 mg/day maintenance dose; Nortriptyline: about 50-200 mg/day maintenance dose; Paroxetine: about 20 to about 50 mg once/day; Phenelzine: about 15-60 mg/day maintenance dose; Reboxetine: about 1 to about 30 mg, once to four times/day; Sertraline: about 20 to about 500 mg once/day; Tranylcypromine: about 30-60 mg/day maintenance dose; Trazodone: about 75-300 mg/day maintenance dose; Venlafaxine: about 10 to about 150 mg once-thrice/day. According to some embodiments, the pharmaceutical composition of the invention comprises reduced amounts of an antidepressant compared to the recommended dose of said antidepressant.

According to some embodiments, the antidepressant is in a dosage ranging from 10 µg to 100 mg. According to some embodiments, the antidepressant is in a dosage ranging from 50 µg to 50 mg. According to some embodiments, the antidepressant is in a dosage ranging from 100 µg to 40 mg. According to some embodiments, the antidepressant is in a dosage ranging from 400 µg to 20 mg. According to some embodiments, the antidepressant is in a dosage ranging from 500 µg to 10 mg. According to some embodiments, the antidepressant is in a dosage ranging from 1 mg to 5 mg. According to additional embodiments, the antidepressant is in a dosage of less than 100 mg. According to additional embodiments, the antidepressant is in a dosage of less than 50 mg. According to additional embodiments, the antidepressant is in a dosage of less than 15 mg, 10 mg, 5 mg, 2 mg or 1 mg. Each possibility represents a separate embodiment of the invention.

According to some embodiments, the pharmaceutical composition comprises less than 90% antidepressant compared to the recommended dose of said antidepressant. According to some embodiments, the pharmaceutical composition comprises less than 80%, 70%, 60%, 50%, or 40% antidepressant compared to the recommended dose of said antidepressant.

The term “pharmaceutical composition” as used herein refers to any composition comprising at least one pharmaceutically active ingredient, formulated such that it facilitates accessibility of the active ingredient to the target organ.

The term “pharmaceutically acceptable” means approved by a regulatory agency of the Federal or a state government or listed in the U.S. Pharmacopeia or other generally recognized pharmacopeia for use in animals, and more particularly in humans.

The term “carrier” as used herein indicates an inactive substance that serves as mechanisms to improve the delivery and the effectiveness of drugs and can be identified by a skilled person in view of the route of administration and related composition formulation.

The term “excipient” as used herein indicates an inactive substance that can be used in various media including solvents, binders or diluents to bulk up formulations that contain active ingredients (thus often referred to as “bulking agents,” “fillers,” or “diluents”), to allow convenient and accurate dispensation of a drug substance when producing a dosage form. Suitable excipients can include any substance that can be used to bulk up formulations with the ibogaine described herein, antidepressant, or both, to allow for convenient and accurate dosage.

According to some embodiments, the solvent is oil. According to other embodiments, the solvent is water. According to certain embodiments, the solvent is alcohol.

The singular forms “a”, “an”, and “the” include plural referents unless the context clearly dictates otherwise. Thus, for example, reference to “an antidepressant” includes one or more of such antidepressant and equivalents thereof known to those skilled in the art, and so forth.

The pharmaceutical compositions can be administered to the patient by any, or a combination, of several routes, such as oral, intravenous, trans-mucosal (e.g., nasal), pulmonary, transdermal, buccal, sublingual, or long-term depot preparation. Solid compositions for oral administration can contain suitable carriers or excipients, such as corn starch, gelatin, lactose, acacia, sucrose, microcrystalline cellulose, kaolin, mannitol, dicalcium phosphate, calcium carbonate, sodium chloride, lipids, alginic acid, or ingredients for controlled slow release. Disintegrators that can be used include, without limitation, micro-crystalline cellulose, corn starch, sodium starch glycolate and alginic acid. Tablet binders that may be used include, without limitation, acacia, methylcellulose, sodium carboxymethylcellulose, polyvinylpyrrolidone (Povidone), hydroxypropyl methylcellulose, sucrose, starch, and ethylcellulose.

Pharmaceutically acceptable excipients used in the composition of the present invention are selected from but not limited to the group of excipients generally known to persons skilled in the art e.g., vehicles, bulking agents, stabilizers, preservatives, surfactants, hydrophilic polymers, solubility enhancing agents such as glycerin, various grades of polyethylene oxides, beta-cyclodextrins like sulfobutylether-betacyclodextrin, transcutol and glycofurol, tonicity adjusting agents, local anesthetics, pH adjusting agents, antioxidants, osmotic agents, chelating agents, viscosifying agents, wetting agents, emulsifying agents, acids, sugar alcohol, reducing sugars, non-reducing sugars and the like, or mixtures thereof.

The pharmaceutical composition can further comprise pharmaceutical excipients including, but not limited to, sodium chloride, potassium chloride, magnesium chloride, sodium gluconate, sodium acetate, calcium chloride, sodium lactate, and the like. The composition, if desired, can also contain minor amounts of sugar alcohols, wetting or emulsifying agents, and pH adjusting agents.

Pharmaceutical compositions for parenteral administration can also be formulated as suspensions of the active compounds. Such suspensions may be prepared as oily injection suspensions or aqueous injection suspensions. For oily suspension injections, suitable lipophilic solvents or vehicles can be used including fatty oils such as sesame oil, or synthetic fatty acids esters such as ethyl oleate, triglycerides or liposomes. Aqueous injection suspensions may contain substances which increase the viscosity of the suspension, such as sodium carboxymethyl cellulose, sorbitol or dextran. Optionally, the suspension may also contain suitable stabilizers or agents which increase the solubility of the compounds, to allow for the preparation of highly concentrated solutions.

According to additional embodiments, the pharmaceutically acceptable excipient comprises at least one of a binder, a filler, a surfactant, an anti-tacking agent a glidant, a disintegrant, a diluent, a tonicity enhancing agent, a wetting agent, a buffering substance, a colorant, an antioxidant, a flavoring agent, a preservative, a stabilizer or any combination thereof. Each possibility represents a separate embodiment.

Suitable binders include, but are not limited to, polyvinylpyrrolidone, opovidone, hydroxypropyl methylcellulose, starch, and gelatin. Each possibility represents a separate embodiment.

Suitable fillers include, but are not limited to, sugars such as lactose, sucrose, mannitol or sorbitol and derivatives therefore (e.g. amino sugars), ethylcellulose, microcrystalline cellulose, and silicified microcrystalline cellulose. Each possibility represents a separate embodiment.

Suitable buffering or pH adjusting agent include, but are not limited to, acidic buffering agents such as short chain fatty acids, citric acid, acetic acid, hydrochloric acid, sulfuric acid and fumaric acid; and basic buffering agents such as tris, sodium carbonate, sodium bicarbonate, sodium hydroxide, potassium hydroxide and magnesium hydroxide. Each possibility represents a separate embodiment.

As used herein, the term “stabilizer” refers to a compound useful for preventing the degradation of an active ingredient, e.g., an ibogaine or antidepressant.

Suitable antioxidants include, but are not limited to, sorbic acid, ascorbic acid, ascorbate, glycine, α-tocopherol, lycopene, butylated hydroxyanisole (BHA), and butylated hydroxytoluene (BHT). Each possibility represents a separate embodiment.

As used herein, the term “antioxidant” refers to a compound and/or a composition useful for preventing oxidation. In one embodiment, an antioxidant protects an active ingredient from “free radicals”.

Suitable flavoring agents include, but are not limited to, sweeteners such as sucralose and synthetic flavor oils and flavoring aromatics, natural oils, extracts from plants, leaves, flowers, and fruits, and combinations thereof. Exemplary flavoring agents include cinnamon oils, oil of wintergreen, peppermint oils, clover oil, hay oil, anise oil, eucalyptus, vanilla, citrus oil such as lemon oil, orange oil, grape and grapefruit oil, and fruit essences including apple, peach, pear, strawberry, raspberry, cherry, plum, pineapple, and apricot. Each possibility represents a separate embodiment.

According to some embodiments, the pharmaceutical composition comprises a pH buffer. According to some embodiments, the pharmaceutical composition comprises citric acid, acetic acid, monosodium phosphate, N-Cyclohexyl-2-aminoethanesulfonic acid, borate, hydrochloric acid, and/or sodium hydroxide.

According to some embodiments, the pharmaceutical composition is formulated as a tablet. Tablets can be made by compression or molding, optionally with one or more accessory ingredients or additives. Compressed tablets are prepared, for example, by compressing in a suitable tableting machine, the active ingredients in a free-flowing form such as a powder or granules, optionally mixed with a binder (e.g., povidone, gelatin, hydroxypropylmethyl cellulose), lubricant, inert diluent, preservative, disintegrate (e.g., sodium starch glycolate, cross-linked povidone, cross-linked sodium carboxymethyl cellulose) and/or surface-active or dispersing agent. The tablets may optionally be coated or scored, and may be formulated so as to provide slow or controlled release of the active ingredients, using, for example, hydroxypropylmethyl cellulose in varying proportions to provide the desired release profile. Tablets may optionally be provided with a coating, such as a thin film, sugar coating, or an enteric coating to provide release in parts of the gut other than the stomach. Processes, equipment, and toll manufacturers for tablet and capsule making are well-known in the art.

According to some embodiments, the pharmaceutical composition is in a solid oral extended-release dosage form.

According to some embodiments, the present invention further provides extended release of the active compounds of, for example, over 4 hours, over 5 hours, over 6 hours, over 7 hours, over 8 hours, over 9 hours, over 10 hours, or more.

According to other embodiments, the pharmaceutical composition is formulated for injectable uses. Injectable compositions may contain various carriers such as vegetable oils, dimethylacetamide, dimethylformamide, ethyl lactate, ethyl carbonate, isopropyl myristate, ethanol, polyols (e.g., glycerol, propylene glycol, and liquid polyethylene glycol). For intravenous injections, the compounds may be administered by the drip method, whereby a pharmaceutical composition containing the active compounds and a physiologically acceptable excipient is infused. Physiologically acceptable excipients may include, for example, 5% dextrose, 0.9% saline, Ringer’s solution or other suitable excipients.

### Methods of Treatment

According to an aspect, the present invention provides a method of treating psychiatric disease or a symptom thereof comprising administering to a subject in need of such treatment a therapeutically effective amount of ibogaine or a derivative thereof and at least one antidepressant.

According an additional aspect, the present invention provides a method of treating neuropsychiatric disease comprising administering to a subject in need of such treatment a therapeutically effective amount of a pharmaceutical composition comprising ibogaine or a derivative thereof, and at least one antidepressant drug, and a pharmaceutically acceptable carrier, excipient or diluent. According to specific embodiments, the neuropsychiatric disease is depression.

According to additional aspect, the present invention provides a method of treating depression, comprising administering to a subject in need of such treatment a therapeutically effective amount of ibogaine or a derivative thereof and at least one antidepressant.

According to specific embodiments, the antidepressant is bupropion. According to certain exemplary embodiments, the ibogaine derivative is noribogaine.

According to additional aspect, the present invention provides a method of treating obesity, comprising administering to a subject in need of such treatment a therapeutically effective amount of ibogaine or a derivative thereof and at least one antidepressant.

The antidepressant and ibogaine or ibogaine derivative are as described hereinabove.

According to some embodiments, the subject is human.

The terms “treat,” “treating,” and “treatment” are meant to include alleviating or abrogating a disorder, or one or more of the symptoms associated with the disorder, and/or alleviating or eradicating the cause(s) of the disorder itself.

The term “therapeutically effective amount” of the compound is that amount of a composition containing the ibogaine and the antidepressant according to the present invention which is sufficient to provide a beneficial effect to the subject to which the compound is administered. An effective amount of the compound may vary according to factors such as the disease or disorder state, age, sex, and weight of the individual.

The term “effective amount” as used herein refers to a sufficient amount of the compositions comprising the ibogaine and the at least one antidepressant to treat or reduce at least one symptom associated with depression.

The term “administering” means delivering a compound (e.g., a combination of iboga and an antidepressant) to a particular organism, such as a human. The term “administering” includes oral administration, inhalation administration, nasal administration, intravenous administration (IV), intraperitoneal administration (IP), transdermal administration, etc.

According to some embodiments, the method is used for treating resistant depression. Treatment-resistant depression includes unipolar depression that does not respond satisfactorily to one or more treatments that are optimally delivered. If the depression has not benefited from at least two adequate trials of medications from different classes in the current episode, clinically significant treatment resistance is present.

Any chronic, treatment-resistant depression may be treated by the methods described herein. Such depression may include but is not limited to any of: major depressive disorder, single episode, recurrent major depressive disorder-unipolar depression, seasonal affective disorder-winter depression, bipolar mood disorder-bipolar depression, mood disorder due to a general medical condition with major depressive-like episode, or mood disorder due to a general medical condition with depressive features, wherein those disorders are resistant to treatment in a given patient.

The methods described herein include administering, in combination, a pharmaceutical combination or composition of the invention as described herein and at least one additional therapy, such as a therapeutic agent selected from the group consisting of at least one anti-anxiety drug, at least one additional anti-depressant drug, at least one neuroleptic medication, at least one mood stabilizer drug, at least one antipsychotic drug, and any combinations thereof. According to additional embodiments, the pharmaceutical combination or pharmaceutical composition is administered in combination with or concurrently with another therapeutic intervention to enhance the efficacy thereof. Examples of other therapeutic interventions include, but are not limited to, counseling, psychotherapy, cognitive therapy or the like, electroconvulsive therapy, hydrotherapy, electrotherapy and electrical stimulation.

The compositions disclosed herein may be administered systemically. According to some embodiments, the pharmaceutical composition is formed into a dosage form suitable for, oral, intravenous, intranasal, intraarterial, or subcutaneous administration.

According to some embodiments the pharmaceutical composition is formulated for an oral use. According to certain embodiments the pharmaceutical composition is formulated for an injectable or inhalation use.

According to some embodiments, the pharmaceutical compositions are administered at least one time a day. According to other embodiments, the compositions are administered 1-4 times a day.

The pharmaceutical combinations or pharmaceutical compositions of the present invention may be administered in combination with other medications for treatment of depression.

According to some embodiments, said compositions are administered in combination with minerals. The minerals are selected from Zinc, Copper, Calcium, Phosphorus, and Silicon. The compositions described herein may further be administered in combination with vitamins. The vitamins are selected from, but not limited to, Vitamin C, Vitamin D, Vitamin E or Vitamin K. Vitamin D may comprise Vitamin D₁, Vitamin D₂, Vitamin D₃, Vitamin D₄, Vitamin D₅ or a combination thereof. Separate dosage forms may be administered simultaneously or sequentially or on entirely independent separate regimens.

According to some embodiments, the pharmaceutical compositions are administered in 2 different steps. First step is a high dosage of ibogaine with the combined drug followed by micro-dose of ibogaine with the combined drug thereafter.

The compositions of the invention may be administered orally in various oral forms including, but not limited to, tablets, capsules, pills, powders, granules, elixirs, tinctures, suspensions, syrups, emulsions and as gel form. In instances in which oral administration is in the form of a tablet or capsule, the composition components can be combined with a non-toxic pharmaceutically acceptable inert carrier or excipients such as lactose, starch, sucrose, glucose, modified sugars, modified starches, methylcellulose and its derivatives, mannitol, sorbitol, and other reducing and non-reducing sugars, magnesium stearate, stearic acid, sodium stearyl fumarate, glyceryl behenate, amorphous silica gel or other desiccant material and the like.

The compositions of the invention may typically be administered in daily doses of from about 300 µg to about 5 mg ibogaine and 0.5 mg to about 15 mg of the antidepressant described herein. According to alternative embodiments, the compositions of the invention may typically be administered in daily doses of from about 0.5 mg to about 50 mg ibogaine and 1 mg to about 10 mg of the antidepressant described herein.

According to some embodiments, the method comprises a step of administering an initial high dose of ibogaine on day 1 followed by administering reduced doses in the following days. According to additional embodiments, the method comprises a step of administering an initial high dose of ibogaine on day 1 or the first 2-7 days, followed by administering reduced doses in the following days.

According to some embodiments, the method comprises a step of administering reduced doses of ibogaine for at least a week. According to some embodiments, the method comprises a step of administering a daily unit dosage form of 500-1000 mg of ibogaine on day 1 or the first 2-7 days followed by maintenance dosages of less than 50 mg/day, 40 mg/day, 30 mg/day, 20 mg/day or 10 mg/day. Each possibility represents a separate embodiment of the invention. According to some specific, the method comprises a step of administering a unit dosage form of 500-1000 mg of ibogaine on day 1 followed by maintenance dosages of less than 50 mg/day.

The term “dose” when refers to ibogaine, refers to a range of ibogaine, ibogaine derivative, or pharmaceutical salt or solvate thereof that provides a therapeutic level of ibogaine when given to a patient in need thereof. The dose is recited in a range, for example from about 1 mg to about 800 mg, and can be expressed either as microgram, milligrams or as mg/kg body weight. The attending clinician will select an appropriate dose from the range based on the patient’s weight, age, degree of addiction, health, and other relevant factors, all of which are well within the skill of the art.

According to some embodiments, the administering of the ibogaine or a derivative thereof and the at least one antidepressant is carried out substantially simultaneously, concurrently, alternately, sequentially or successively. In some embodiments, the ibogaine or a derivative thereof and the at least one antidepressant are administered according to overlapping schedules. According to specific embodiments, the ibogaine or a derivative thereof and the at least one antidepressant are administered simultaneously. According to some embodiments, the ibogaine or derivative thereof are administered concurrently with the antidepressant. According to additional exemplary embodiments, the administering of ibogaine or a derivative thereof and the at least one antidepressant is carried out sequentially.

As used herein “administered simultaneously”, or “simultaneous administration”, refers to therapy in which the both agents (e.g., ibogaine and antidepressant) are administered at the same time, suitably as a mono-therapy.

As used herein “sequential administration” means that one agent is administered after the other, however, the time period between the administration of each agent is such that both agents are capable of acting therapeutically concurrently. Thus, administration “sequentially” may permit one agent to be administered within seconds, minutes, or a matter of hours after the other provided the circulatory half-life of the first administered agent is such that they are both concurrently present in therapeutically effective amounts.

According to additional embodiments, the administering of ibogaine or a derivative thereof and the at least one antidepressant is carried out separately

As used herein, “separate administration” means that one agent is administered after the other, however, the time period between administration is such that the first administered agent is no longer present a therapeutically effective amount when the second agent is administered. Accordingly, the two agents exert their therapeutic effects separately. Nevertheless, the overall therapeutic effect observed when the two agents separately act therapeutically is significantly greater than either agent used alone.

The following examples are presented in order to more fully illustrate some embodiments of the invention. They should, in no way be construed as limiting the scope of the invention.

### EXAMPLES

### Example 1: Safety and Therapeutically Effects of a Combination of Ibogaine And Antidepressant in Preclinical Experiments

To examine safety, side effects and efficiency of the pharmaceutical combinations described herein, animal models of depression, obesity, PSTD, and anxiety are used. The animals (rats or mice) are treated with antidepressants such as norepinephrine-dopamine reuptake inhibitor (NDRI) or selective serotonin reuptake inhibitor (SSRI), alone or in combination with ibogaine (0.01-4 mg/kg i.p.), ibogaine alone or saline. The animals are administered once daily for 2-60 days.

Animals are monitored for weight, sleep, sexual behavior, locomotion, etc., before, during and after the treatment (with follow-up of additional 30 days posttreatment). Specifically, animal models of depression (and anxiety) are examined for behavioral and/or genetic models (e.g., unpredictable chronic mild stress, Flinders sensitive rat line) + behavioral testing (e.g., sucrose preference, forced swim test, tail suspension test, elevated plus maze, morris water maze).

Additional groups are exposed to extended period of repeated administrations of the combination (> 60 days).

### Example 2: Effect of the Combination of Ibogaine and Bupropion or Prozac On depression

The efficacy and safety of the combination of ibogaine and antidepressants in treating depression is determined by evaluating the short-term effects of the compounds in the treatment of acute unipolar depression.

A prospective, double-blind, active-controlled, randomized clinical trial (RCT) of 4 weeks of fixed doses is conducted in outpatients with unipolar depression. 1, 5, 10, or 20 mg of Bupropion or Prozac daily, alone or in combination with 0.01-2.0 mg/kg ibogaine. Thereafter, all subjects continue to take Bupropion or Prozac daily alone for additional 4 weeks of follow-up, with physiological and psychological evaluations conducted during weekly visits. The patients are not on any other psychotropic medications. Clinical status is assessed using the Hamilton Depression Rating Scale 21 Item (HAMD-21), the Clinical Global Impression Severity and Improvement Scales (CGI-I, CGI-S) and the Hamilton Anxiety Rating Scale (HAM-A) at each clinical visit. Patients complete the Quick Diagnostic Interview Schedule (QDIS) and the Satisfaction of Life Scale at each clinical visit.

Independent t-test analyses are conducted for baseline and endpoint (LOCF) values. Analysis of Variance (ANOVA) are conducted on ratings scales in relationship to time.

The primary endpoint is the change in the total score on the HDRS-21 from baseline to week 4. The secondary efficacy endpoints are response and remission rates, defined as a reduction of at least 30% in the total HDRS-21 score at week 4 compared to baseline and a total HDRS-21 score < 10 at week 4, respectively. Secondary endpoints are the change in HDRS-21 score at the end of follow-up, changes to scores of the other psychological evaluations at week 4 and the end of follow-up, time till beneficial antidepressant effects, and the occurrence of adverse effects.

Side effects including nausea, dizziness, and constipation are reported and analyzed. Additional side effects examined include dry mouth, headaches, diarrhea, and stomach ache.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1-30. (canceled)

31. A pharmaceutical combination comprising: (1) ibogaine or a derivative thereof; and (2) at least one antidepressant.

32. The pharmaceutical combination of claim 31, wherein the ibogaine derivative is selected from the group consisting of noribogaine, dihydroxyibogamine, dihydrocatharanthine, coronaridine, conopharyngine, conoflorine, catharanthine, iboxygaine, iboluteine, ibogamine, ibogaline, ibogaine, epiibogamine, isovoacangine, isovoacristine, 18-methoxycoronaridine (18-MC), kisantin, montanin, tabernanthine, tubotaiwine, voacristine, voacangine, voaluteine, and voacamine.

33. The pharmaceutical combination of claim 32, wherein the ibogaine derivative is noribogaine.

34. The pharmaceutical combination of claim 31, wherein the antidepressant is selected from the group consisting of monoamine oxidase (MAO) inhibitor, tricyclic antidepressant, serotonin reuptake inhibitor, selective norepinephrine reuptake inhibitors (SNRIs), aminoketones, serotonin antagonists, dopamine reuptake inhibitors, dual reuptake inhibitors, norepinephrine enhancers, serotonin activity enhancers, dopamine activity enhancers, and any combination thereof.

35. The pharmaceutical combination of claim 31, wherein the antidepressant is selected from the group consisting of bupropion, fluoxetine, amitriptyline, clomipramine, desipramine, doxepin, imipramine hydrochloride, imipramine pamoate, maprotiline, nortriptyline, protriptyline, trimipramine, citalopram, escitalopram, moclobemide, fluvoxamine, paroxetine, sertraline, nefazodone, and trazodone.

36. The pharmaceutical combination of claim 31, wherein the combination comprises ibogaine or derivative thereof in a dosage ranging from 1 mg to 750 mg.

37. The pharmaceutical combination of claim 31, wherein the combination comprises an antidepressant in a dosage ranging from 10 ug to 100 mg.

38. The pharmaceutical combination of claim 31, wherein said pharmaceutical combination is formulated in a dosage form selected from the group consisting of tablet, caplet, pill, capsule, pellets, granules, powder, lozenge, sachet, cachet, elixir, suspension, gel and dispersion.

39. A method of treating psychiatric disease or disorder comprising administering to a subject in need of such treatment a pharmaceutical combination according to claim 31.

40. The method of claim 39, wherein the psychiatric disease or disorder is depression.

41. The method of claim 40, wherein the depression is a treatment resistant depression (TRD).

42. The method of claim 39, wherein the treatment has no or minimal dissociative side effects upon administration to a subject.

43. The method of claim 39, wherein the treatment reduces a depression symptom selected from the group consisting of neuropathic pain, sexual dysfunction, hopelessness, helplessness, anxiety, worries, memory problems, obesity, cognitive impairment, loss of feeling of pleasure (anhedonia), slowed movement, irritability, and lack of interest in personal care.

44. The method of claim 39, wherein the treatment reduces a side effect selected from the group consisting of nausea, vomiting, dizziness, insomnia, sleepiness, trouble sleeping, abnormal dreams, constipation, sweating, dry mouth, yawning, tremor, gas, anxiety, agitation, abnormal vision, headache, and sexual dysfunction.

45. The method of claim 39, wherein the treatment reduces the time until a clinical effect is shown.

46. The method of claim 39, wherein the ibogaine or derivative thereof and the antidepressant are present within a single pharmaceutical composition.

47. The method of claim 39, the method comprises administering the pharmaceutical composition or combination at a daily dose comprising from about 0.1 to about 500 mg/day ibogaine and from about 0.5 to about 50 mg/day of an antidepressant.

48. The method of claims 39, comprising a step of administering a high dose of ibogaine on day 1 followed by administering reduced doses in the following days.

49. The method of claim 39, comprising a step of administering a unit dosage form of 500-1000 mg of ibogaine on day 1 followed by maintenance dosages of less than 50 mg/day.

50. The method of claim 39, wherein the antidepressant is administered in reduced amount compared to the standard dose.
